(19) United States
(12) Patent Application Publication
(10) Pub. No.: US 2005/0074406 A1
(43) Pub. Date: Apr. 7, 2005

(54) Ultrasound coating for enhancing visualization of medical device in ultrasound images

(75) Inventors: Lucien Alfred Couvillon JR., Concord, MA (US); Tat Jin Teo, Sunnyvale, CA (US)

Correspondence Address: David E. Wang;Orrick, Herrington & Sutcliffe LLP, 4 Park Plaza, Suite 1600, Irvine, CA, 92614-2558, US

(73) Assignee: SCIMED LIFE SYSTEMS, INC.

(21) Appl. No.: 10/678,337

(22) Filed: Oct. 3, 2003

(51) Int. Cl. A61K 49/00; A61B 8/00

(52) U.S. Cl. 424/9.5; 600/431

(57) ABSTRACT

The invention enhances ultrasonic visualization of a medical device by coating the surface of the medical device with a contrast agent preferably comprising microbubbles. The contrast agent responds to incident ultrasound waves to generate vibrations at a harmonic frequency (i.e., a harmonic of the frequency of the incident wave), non-harmonic vibrations or a combination of harmonic and non-harmonic vibrations. In one embodiment, the microbubbles are coated on the medical device using an intermediate adhesion layer that adheres to the surface of the device. In a second embodiment, the microbubbles are incorporated in the adhesion layer.

### FIELD OF THE INVENTION

The field of the invention relates to contrast agents for ultrasound imaging medical devices, and more particularly, to coating medical devices with ultrasound contrast agents to enhance visualization of the medical devices in ultrasound images.

### BACKGROUND OF THE INVENTION

Catheters, needles and many other devices are used in minimally-invasive medical procedures to gain access to the interior of the body. The catheters typically include working elements (e.g., electrodes, forceps, snares, angioplasty balloons, stents, and transducers) at or near their distal tip for performing medical procedures within the internal organs and/or orifices of the body for injecting fluids.

In radio frequency (RF) ablation procedures, for example, a catheter equipped with an RF electrode(s) is guided to position the RF electrode at a localized region of the body to be ablated. After the RF electrode is positioned, the RF electrode is energized to emits RF energy to the localized region, which heats and destroys the tissue in the localized region. RF ablation is effective in the treatment of arrhythmias by selective ablation of diseased heart tissue responsible for abnormal electrical conduction in the heart. Recently, RF ablation has become popular in the treatment of various tumors (e.g., of the liver, breast, brain and bone).

Imaging techniques, such as ultrasound, fluoroscopy, magnetic resonance imaging (MRI) and CT, are commonly used to guide less-invasive catheters inside the body. In ablation procedures, for example, ultrasound imaging is used to position RF electrodes at the region of the body to be ablated. Ultrasound imaging guidance is attractive because the modality is simpler and less expensive than other modalities, and does not involve exposure to ionizing radiation. However, a limitation of conventional ultrasound imaging is that electrodes are poorly visualized, which inhibits the ability of physicians to properly position the electrodes in the body. Various techniques have been proposed to enhance the visibility of electrodes in ultrasound images, including roughening the surface of the electrodes. These techniques enhance visibility by increasing the electrodes' ability to reflect or backscatter incident ultrasound waves.

A problem with these techniques is that the surrounding tissue also backscatters incident ultrasound waves. As a result, the contrast between the electrodes and the surrounding tissue can be poor, making it difficult for physicians to distinguish the electrodes from the background in conventional ultrasound images.

In recent years, an ultrasound imaging technique known as harmonic ultrasound imaging has been developed to enhance the visualization of blood flow in the body by suppressing the background image. This technique uses contrast agents, usually comprised of microbubbles. When illuminated by an ultrasound wave at frequency f, the contrast agents reradiate the ultrasound waves nonlinearly, and generate vibrations including harmonic frequencies, 2f, 3f, . . . due to the microbubbles' nonlinear response to the incident wave. For example, a “square-law” contrast agent reradiates an ultrasound wave at twice the frequency of the incident wave. Other types of nonlinearities similarly produce other harmonics at other frequencies and amplitudes, and excited vibrational modes in the microbubbles can produce subharmonics as well. [[subharmonics might be invention, not in prior art]]

To generate a harmonic ultrasound image, the contrast agent is usually injected intravenously into the patient's veins. An ultrasound transmitter then illuminates the target region of the body with ultrasound waves at a fundamental frequency. The contrast agent carried along with the blood reradiates the ultrasound waves at a harmonic frequency (e.g., twice the fundamental frequency) while the surrounding body tissue backscatters the incident ultrasound waves mostly at the fundamental frequency. As a result, an ultrasound imaging system tuned to the harmonic frequency receives an enhanced signal from the contrast agent in the blood and a diminished signal from the surrounding tissue. This enables the imaging system to generate a harmonic ultrasound image in which the contrast agent in the blood appears bright and the background body appears dim, thereby enhancing visualization of the blood containing the contrast agent.

### SUMMARY OF THE INVENTION

The invention enhances ultrasonic visualization of a medical device by providing upon the surface of the medical device a contrast agent, preferably comprising microbubbles, that interacts with the illuminating ultrasound differently than does the surrounding tissue. The contrast agent may be a substance having acoustic properties that are substantially different than those of the surrounding tissue such as, for example, agents that have an acoustic impedance or attenuation significantly different than that of water or tissue.

The applied contrast agent may reradiate incident ultrasound waves at a harmonic frequency (i.e., a multiple of the fundamental frequency of the incident signal). Alternatively, the contrast agent may be reradiate ultrasound waves at a non-harmonic frequency or a combination of harmonic and non-harmonic frequencies.

In one example embodiment, the contrast agent is coated upon the surface of the medical device using an intermediate adhesion or binding layer that adheres to the surface of the medical device. The adhesion layer may comprise a pressure sensitive adhesive that strongly adheres to the surface of the medical device when wetted, e.g., by blood in the body. Examples of pressure sensitive adhesives that can be used include silicone, polymer and hydrogel.

In another embodiment, the microbubbles may be imbedded within an absorbent outer layer coated over the contrast agent. The outer layer may comprises polymer, elastomer, or hydrogel to provide a smooth and lubricious outer surface for the medical device. The outer layer may also comprise therapeutic agents.

In still another embodiment, the contrast agent is incorporated in the adhesion layer.

Other aspects of the invention will be or will become apparent to one with skill in the art upon examination of the following figures and detailed description.

### BRIEF DESCRIPTION OF THE DRAWINGS

The components in the figures are not necessarily to scale, emphasis instead being placed upon illustrating the principles of the invention.

FIG. 1 illustrates an ultrasound coating according to one embodiment, in which an ultrasound contrast agent is coated on a medical device using an intermediate adhesion layer.

FIG. 2 illustrates the ultrasound coating of FIG. 1 further comprising an outer layer coated over the contrast agent.

FIG. 3 illustrates an ultrasound coating according to another embodiment, in which the ultrasound contrast agent is incorporated in the adhesion layer.

FIG. 4 illustrates a medical procedure utilizing the improved ultrasound coating to visualize a medical device inside the body.

### DETAILED DESCRIPTION OF THE INVENTION

The invention enhances ultrasonic visualization of a medical device by coating the surface of a medical device with an ultrasound contrast agent. The medical device may be an insertable device, e.g., catheter or guidewire, adapted to perform medical procedures inside the body. The medical device may also be a prosthesis, e.g., stent, adapted to be implanted in the body. The contrast agent should have good contrast properties, i.e., a significant difference in acoustic impedance from the surrounding material.

In one embodiment, the contrast agents are harmonic ultrasound contrast agents that reradiate incident ultrasound waves nonlinearly and generate vibrations at a harmonic frequency (i.e., a harmonic of the frequency of the incident wave). When used in conjunction with an ultrasound imaging system tuned to the harmonic frequency, the contrast agents enhance visualization of the coated medical device by making the device stand out from the background in the harmonic ultrasound image. This is because the backscattered waves at the harmonic frequency arise almost entirely from the contrast agents on the medical device, and not the surrounding body.

Alternatively, the contrast agents may be non-harmonic ultrasound contrast agents that backscatter ultrasound waves nonlinearly and generate vibrations at substantially the fundamental frequency of the incident waves. Such contrast agents would enhance the visualization of the coated medical device when imaged by a conventional ultrasound imaging system tuned to the fundamental frequency. The contrast agent can also comprise a combination of harmonic and non-harmonic contrast agents so that the coated device can be visualized by ultrasound imaging systems tuned to harmonic or fundamental frequencies.

The contrast agent may also be sub-harmonic ultrasound contrast agent that reradiates incident ultrasound waves at a sub-harmonic frequency (i.e., a sub-harmonic of the frequency of the incident wave). The sub-harmonic contrast agent can accomplish the same purpose as the harmonic contrast agent, which is to make the coated medical device stand out from the background in the ultrasound image. This is because the sub-harmonic contrast agent reradiates incident ultrasound waves at the sub-harmonic frequency while the surrounding body tissue backscatters the ultrasound waves at the fundamental frequency. This enables an ultrasound imaging system tuned to the sub-harmonic frequency to detect the ultrasound waves from the coated medical device while ignoring the backscattered ultrasound waves from the surrounding body.

Thus, these harmonics may include higher integral harmonics (e.g., 2f, 3f, 4f) as well as integral subharmonics (e.g., f/2, f/3, f/4). Noninteger harmonics and subharmonics as 1.5f and f./1.5 are not generated.

Another method to suppress the background image is to coat the medical device with a non-linear contrast agent and emit an additional reversed phase ultrasound wave into the body. The more linear surrounding body tissue will reflect the same reversed phase waveform and provide a better cancellation than that from the more non-linear contrast agent on the medical device. This results in a cancellation of the background tissue, leaving a clearer image of the device coated with the contrast agent. The non-linear contrast agent may be a harmonic or sub-harmonic contrast agent since both are non-linear.

In addition to visualizing medical devices, the improved contrast agent is useful for enhancing visualization of elements of medical devices. For example, the contrast agents can be coated on the electrodes of a needle electrode catheter to enhance visualization of the electrodes. This would facilitate the guidance and placement of the electrodes at a treatment site inside the body.

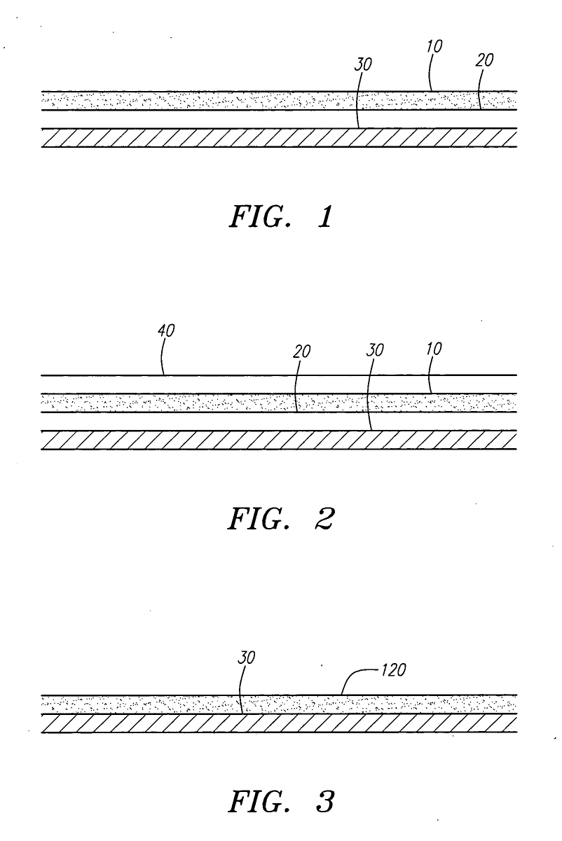

FIG. 1 illustrates an example embodiment of the invention in which the ultrasound contrast agent 10 is coated on a medical device using an intermediate adhesion layer 20 that adheres to the surface 30 of the device. The surface 30 can be a metal or plastic surface or a combination thereof. For example, the surface 30 can be the metal surface of an electrode or the plastic surface of a catheter body.

The contrast agent 10 preferably comprises microbubbles, each microbubble further comprising a generally spherical membrane encapsulating a gas-filled core. The membrane may comprise silicone, polymer, cellulose or other biocompatible material, and the gas-filled core may comprise CO², O², N², room air, fluorocarbons or other suitable gas. A gas-filled core is not required as long as there is a large difference in acoustic impedance between the microbubbles and the surrounding body so that the microbubbles strongly backscatter ultrasound energy in the body. The membrane and core permit the microbubbles to vibrate in response to incident ultrasound waves and generate nonlinear vibrations at harmonic and/or subharmonic frequencies.

In one embodiment, the microbubbles reradiate incident ultrasound waves at a frequency that is a harmonic of the frequency of the incident wave. Harmonic ultrasound microbubbles are known in the art and are commercially available from various pharmaceutical companies, e.g., Alliance Pharmaceutical Corp. Alternatively, the microbubbles can be non-harmonic microbubbles or a combination of harmonic and non-harmonic microbubbles.

In one embodiment, an adhesion layer 20 adheres the microbubbles of the contrast agent 10 to the surface 30 of the medical device. The adhesion layer 20 may be a pressure sensitive adhesive that strongly adheres to the surface 30 of the medical device when wetted, e.g., by blood in the body. Examples of pressure sensitive adhesives include silicone, polymers, hydrogels, and the like. An outer layer of a polymer, elastomer, or hydrogel may be added over the adhesion layer 20, where the resulting encapsulation of the microbubbles defines the microbubble concentration. Hydrogels may also be used, as is commonly so, as a coating on the surface of medical devices to make the devices smoother and more lubricious. This is done to prevent the devices from causing tissue damage when inserted into the body. Additional examples of pressure sensitive adhesives that may be used can be found in U.S. Pat. Nos. 6,316,522; 6,261,630; 6,184,266; 6,176,849; 6,096,108; 6,060,534; 5,702,754; 5,693,034; and, 5,304,121, each of which is incorporated herein in its entirety by reference.

Another suitable adhesive for the adhesion layer 20 is DOPA (dihydroxyphenylalanine), an amino acid that adheres well to metal and plastic surfaces, even when the surfaces are wet. DOPA is naturally found in mussel adhesive protein, a sticky glue that keeps common mussels (Mytilus edulis) firmly anchored to rocks and other objects, allowing them to withstand the extreme pounding of ocean waves.

The contrast agent 10 overlays the adhesion layer 20. The microbubbles in the contrast agent 10 physically sit on top of the adhesion layer 20 and may adhere to the adhesive layer 20 via intermolecular forces, e.g., hydrogen bonding, Van der Waals forces and/or physical interlocks. Intermolecular forces are commonly used to adhere therapeutic agents, e.g., heparins, onto pressure sensitive adhesives. For example, pressure sensitive adhesives, e.g., hydrogels, absorb moisture when wetted, e.g., by blood in the body. The absorbed moisture attracts particles having hydrophilic surfaces to the pressure sensitive adhesives via hydrogen bonding. Because microbubbles typically have hydrophilic outer surfaces, similar forces can adhere the microbubbles to the adhesion layer 20.

The adhesion layer 20 may be coated onto the surface 30 of the medical device by dissolving the adhesive in a solvent to produce an adhesion solution, and applying the adhesion solution to the surface 30. The solution may be sprayed or brushed onto the surface 30 or the surface 30 may be dipped into the solution. After the adhesion solution is applied to the surface 30, the surface 30 is dried to remove the solvent in the solution and form the adhesion layer 20. This coating technique is commonly used to coat pressure sensitive adhesives, e.g., polymers and hydrogels, onto medical devices.

The microbubbles of the contrast agent 10 may then be coated onto the adhesion layer 20 by preparing the microbubbles in an emulsion solution, and applying the emulsion solution to the adhesion layer 20. The emulsion solution may be sprayed or brushed onto the adhesion layer 20 or the adhesion layer 20 may be dipped into the emulsion solution.

FIG. 2 illustrates an embodiment in which an outer layer 40 is coated over the contrast agent 10, thereby sandwiching the contrast agent 10 between the outer layer 40 and the adhesion layer 20. The outer layer 40 may comprise a hydrogel or polymer to provide a smooth and lubricious outer surface for the medical device. Therapeutic agents may be incorporated into the hydrogel or polymer for time release of the therapeutic agents into the body. Examples of hydrogels incorporating therapeutic agents can be found in U.S. Pat. No. 5,843,089, which is hereby incorporated in its entirety by reference.

FIG. 3 illustrates another embodiment in which the microbubbles are incorporated in the adhesion layer 120. The microbubbles may be entrapped in the adhesion layer 120 by mixing the microbubbles into an adhesion solution, applying the solution to the surface 30 of the medical device, and drying the solution to form the adhesion layer 120. For example, hydrogels may be used for the adhesion layer 120 because of their ability to form a dense matrix that can entrap microbubbles. In addition to microbubbles, other particles may be entrapped in the adhesion layer 120 such as therapeutic agents. Furthermore, an additional layer (not shown) may be coated over the adhesion layer 120 if desired. This layer may comprise additional microbubbles, therapeutic agents, polymers, hydrogels or the like.

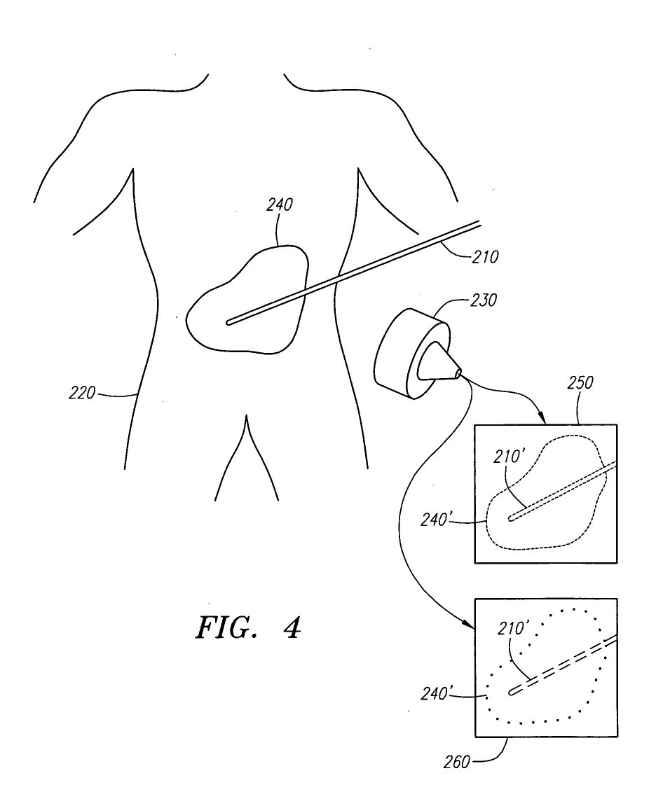

FIG. 4 illustrates a medical procedure utilizing the improved ultrasound contrast agent coating. In this example, a harmonic ultrasound contrast agent is coated onto a needle electrode catheter 210, which is inserted into the body 220 to perform the medical procedure, e.g., tissue ablation. The catheter 210 is visualized inside the body 220 by an ultrasound imaging system comprising an ultrasound transducer 230. The ultrasound transducer 230 emits ultrasound waves at a fundamental frequency into the body 220. The ultrasound transducer 230 detects the backscattered ultrasound waves to generate an ultrasound image of the catheter 210 and the surrounding body 220.

Ultrasound image 250 illustrates an exemplary ultrasound image in which the transducer 230 is tuned to detect backscattered ultrasound waves at the fundamental frequency. In this image 250, the catheter image 210′ appears dim and there is low contrast between the catheter image 210′ and surrounding body tissue image 240′. This is because the contrast agent coating on the catheter 210 reradiates the incident ultrasound waves at a harmonic frequency, which is not detected by the transducer 230 tuned to the fundamental frequency.

Ultrasound image 260 illustrates an exemplary ultrasound image in which the transducer 230 is tuned to detect backscattered ultrasound waves at the harmonic frequency of the contrast agent. In this image 260, the catheter image 210′ appears bright and the surrounding body tissue image 240′ appears dim. This is because the backscattered waves at the harmonic frequency arise almost entirely from the contrast agent coating of the catheter 210. The enhanced visualization of the catheter 210 in the harmonic ultrasound image enables a physician to more easily and accurately guide the needle catheter 210 inside the body.

While the invention is susceptible to various modifications and alternative forms, a specific example thereof has been shown in the drawings and is herein described in detail. For example, each feature of one embodiment can be mixed and matched with other features shown in other embodiments. Features and processes known to those of ordinary skill in the art of medical devices and/or contrast agents may similarly be incorporated as desired. Additionally and obviously, features may be added or subtracted as desired. It should be understood, however, that the invention is not to be limited to the particular form disclosed, but to the contrary, the invention is to cover all modifications, equivalents, and alternatives falling within the spirit and scope of the appended claims.

1. An ultrasound coating for enhancing ultrasonic visualization of a medical device, comprising
an adhesion layer adhering to a surface of the medical device; and
a contrast agent layer overlaying the adhesion layer, the contrast agent layer comprising ultrasound microbubbles.

2. The ultrasound coating of claim 1, wherein the microbubbles vibrate in response to an incident ultrasound wave to generate an harmonic vibration.

3. The ultrasound coating of claim 1, wherein the microbubbles vibrate in response to an incident ultrasound wave to generate a non-harmonic vibration.

4. The ultrasound coating of claim 1, wherein the microbubbles vibrate in response to an incident ultrasound wave to generate both harmonic and non-harmonic vibrations.

5. The ultrasound coating of claim 1, wherein the microbubbles vibrate in response to an incident ultrasound wave to generate a sub-harmonic vibration.

6. The ultrasound coating of claim 1, wherein the adhesion layer comprises a pressure sensitive adhesive.

7. The ultrasound coating of claim 1, wherein the adhesion layer comprises silicone.

8. The ultrasound coating of claim 1, wherein the adhesion layer comprises polymer.

9. The ultrasound coating of claim 1, wherein the adhesion layer comprises hydrogel.

10. The ultrasound coating of claim 1, wherein the adhesion layer comprises DOPA (dihydroxyphenylalanine).

11. The ultrasound coating of claim 1, further comprising a third layer overlaying the contrast agent layer.

12. The ultrasound coating of claim 11, wherein the third layer comprises polymer or hydrogel.

13. The ultrasound coating of claim 12, wherein the third layer comprises therapeutic agents.

14. An ultrasound coating for enhancing ultrasonic visualization of a medical device, comprising:
an adhesion layer adhering to the surface of the medical device; and
ultrasound microbubbles in the adhesion layer.

15. The ultrasound coating of claim 14, wherein the microbubbles vibrate in response to an incident ultrasound wave to generate a harmonic vibration.

16. The ultrasound coating of claim 14, wherein the microbubbles vibrate in response to an incident ultrasound wave to generate a non-harmonic vibration.

17. The ultrasound coating of claim 14, wherein the microbubbles vibrate in response to an incident ultrasound wave to generate both harmonic and non-harmonic vibrations.

18. The ultrasound coating of claim 14, wherein the microbubbles vibrate in response to an incident ultrasound wave to generate a sub-harmonic vibration.

19. The ultrasound coating of claim 14, wherein the adhesion layer comprises a pressure sensitive adhesive.

20. The ultrasound coating of claim 14, wherein the adhesion layer comprises silicone.

21. The ultrasound coating of claim 14, wherein the adhesion layer comprises polymer.

22. The ultrasound coating of claim 14, wherein the adhesion layer comprises hydrogel.

23. The ultrasound coating of claim 14, wherein the adhesion layer comprises DOPA (dihydroxyphenylalanine).

24. The ultrasound coating of claim 14, further comprising therapeutic agents in the adhesion layer.

25. A medical device adapted to be inserted into a patient's body, the medical device having at least a portion coated by the ultrasound coating of claim 1.

26. A medical device adapted to be inserted into a patient's body, the medical device having at least a portion coated by the ultrasound coating of claim 13.

27. The medical device of claim 25 wherein the medical device is a stent.

28. The medical device of claim 25 wherein the medical device is a catheter.

29. The medical device of claim 25 wherein the medical device is a prosthesis.

30. The medical device of claim 26 wherein the medical device is a stent.

31. The medical device of claim 26 wherein the medical device is a catheter.

32. The medical device of claim 26 wherein the medical device is a prosthesis.
